(19) United States
(12) Patent Application Publication
(10) Pub. No.: US 2026/0207341 A1
(43) Pub. Date: Jul. 23, 2026

(54) TOTAL ANKLE REPLACEMENT WITH VARIABLE CONSTRAINT ARTICULATING SURFACES

(71) Applicant: Enovis Corporation, Lewisville, TX (US)

(72) Inventors: Samuel Fang, Plano, TX (US); Matthew David Schultz, Addison, TX (US); Hannah Fatemi, Carrollton, TX (US)

(21) Appl. No.: 19/450,927

(22) Filed: Jan. 16, 2026

Related U.S. Application Data
(60) Provisional application No. 63/746,380, filed on Jan. 17, 2025.

(51) Int. Cl. A61F 2/42; A61F 2/30

(52) U.S. Cl. CPC A61F 2/4202; A61F 2002/30327; A61F 2002/30518; A61F 2002/4207

(57) ABSTRACT

A total ankle replacement implant with a variable constraint bearing surface is provided. The variable constraint bearing surface may have regions of high constraint to provide the total ankle replacement with stability when stationary and regions of low constraint to improve the range of motion and internal-external rotation of the total ankle replacement implant to improve kinematics during flexion and extension.

### CROSS REFERENCE TO RELATED APPLICATIONS

This application claims priority to and the benefit of U.S. Provisional Patent Application No. 63/746,380, filed on Jan. 17, 2025, which is hereby incorporated by reference in its entirety.

### FIELD

This disclosure relates to a total ankle replacement device. More specifically, this disclosure relates to articulating surfaces for a talus component of a total ankle replacement implant.

### BACKGROUND

Total ankle replacement is a procedure that is used for patients with, for example, osteoarthritis, post-traumatic arthritis, or rheumatoid arthritis affecting the ankle joint. Early total ankle replacement implants were designed with bearing surfaces that mimicked the biological geometry of the ankle joint, but eventually evolved to have bicondylar bearing surfaces with reciprocal articulation surfaces engaged therewith. While these bicondylar bearing surfaces increased the stability of ankle replacement implants, compared to conventional designs, these implants limit the range of motion during flexion and extension of the ankle joint, which can not only cause patient discomfort but may also lead to premature wear of the bearing surface. These implants, for example, may have a uniform condyle radius that is particularly limiting during high flexion (i.e., dorsiflexion) articulation.

It is therefore desirable to provide bearing surfaces for total ankle replacement implants that increase range of motion during flexion while otherwise maintaining stability.

### SUMMARY

In one aspect, a talar component for a total ankle replacement implant is provided. The talar component may include a bearing surface having a high constraint region and a low constraint region, where the high constraint region and the low constraint region of the bearing surface define a range of motion for the total ankle replacement implant. In some aspects, the high constraint region defines a first radius, the first radius being a distance between the bearing surface and an opposed bottom surface of the talar component at a maximum height of the bearing surface, and the low constraint region defines a second radius, the second radius between the bearing surface and the opposed bottom surface of the talar component at a minimum height of the bearing surface. A ratio of the first radius to the second radius is between 2:1 to 1.5:1.

In some aspects, the bearing surface is configured to maximize contact between the high constraint region of the bearing surface and an articulating surface configured to engage with and move relative to the bearing surface when the total ankle replacement is in a neutral position. In some aspects, the bearing surface is configured to minimize contact between the low constraint region of the bearing surface and an articulating surface configured to engage with and move relative to the bearing surface when the total ankle replacement is in a flexed position or an extended position. In the flexed position or the extended position, the talar component may be capable of internal rotation and external rotation relative to the articulation surface.

In another aspect, a total ankle replacement implant is provided. The total ankle replacement implant may include an articulation component having an articulation surface and a talar component having a bearing surface, where the bearing surface has a high constraint region and a low constraint region defining a range of motion of the talar component relative to the articulation component. In some aspects, the articulation surface is concave and defines a uniform radius of curvature. In some aspects, the bearing surface is convex and configured to be received within the articulation surface. In some aspects, the high constraint region defines a first radius, the first radius being a distance between the bearing surface and an opposed bottom surface of the talar component at a maximum height of the bearing surface, and the low constraint region defines a second radius, the second radius being a distance between the bearing surface and the opposed bottom surface of the talar component at a minimum height of the bearing surface. The first radius may be greater than the second radius.

In some aspects, the bearing surface is configured to maximize contact between the high constraint region of the bearing surface and the articulating surface when the total ankle replacement is in a neutral position. In some aspects, the bearing surface is configured to minimize contact between the low constraint region of the bearing surface and the articulating surface when the total ankle replacement is in a flexed position or an extended position. In the flexed position or the extended position, the talar component may be capable of internal rotation and external rotation relative to the articulation surface. The articulation surface may include a lateral articulation surface configured to receive the lateral bearing surface, and a medial articulation surface configured to receive the medial bearing surface.

In a further aspect, a bearing surface of a talar component for a total ankle replacement implant is provided. The bearing surface may include a high constraint region defining a first radius and a low constraint region defining a second radius, where the first radius is a distance between the bearing surface and an opposed bottom surface of the talar component at a maximum height of the bearing surface, and the second radius is a distance between the bearing surface and the opposed bottom surface of the talar component at a minimum height of the bearing surface. In some aspects, the first radius is greater than the second radius. In some aspects, the high constraint region and the low constraint region define a range of motion of the total ankle replacement implant.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale.

FIG. 1 is a perspective view of a total ankle replacement implant, according to one or more embodiments of the present disclosure.

FIG. 2A is a perspective view of an articulation component of the total ankle replacement implant of FIG. 1, according to one or more embodiments of the present disclosure.

FIG. 2B is a front view of the articulation component of FIG. 2A, according to one or more embodiments of the present disclosure.

FIG. 3A is a perspective view of the talar component of the total ankle replacement implant of FIG. 1, according to one or more embodiments of the present disclosure.

FIG. 3B is a side view of the talar component of FIG. 3A, according to one or more embodiments of the present disclosure.

FIG. 4A is a cross section of the talar component of FIG. 3A across the X-X axis, according to one or more embodiments of the present disclosure.

FIG. 4B is a cross section of the talar component of FIG. 3A across the Y-Y axis, according to one or more embodiments of the present disclosure.

FIG. 5A is a side view of the articulation component and the talar component of the total ankle replacement of FIG. 1 without flexion, according to one or more embodiments of the present disclosure.

FIG. 5B is a cross-sectional front view of the articulation component and the talar component of FIG. 5A, according to one or more embodiments of the present disclosure.

FIG. 6A is a side view of the articulation component and the talar component of the total ankle replacement of FIG. 1 with maximum flexion, according to one or more embodiments of the present disclosure.

FIG. 6B is a cross-sectional front view of the articulation component and the talar component of FIG. 6A, according to one or more embodiments of the present disclosure.

### DETAILED DESCRIPTION

The disclosure will be described more fully hereinafter with reference to the accompanying drawings, in which example embodiments of the disclosure are shown. This disclosure may, however, be embodied in many different forms and should not be limited to the embodiments set forth herein.

A total ankle replacement implant is provided, the total ankle replacement implant having a bicondylar talar component with a variable radius articulating surface. The variable radius of the articulating surface may function to improve the stability and range of motion of the total ankle replacement implant, while also increasing the longevity thereof. For example, the variable radius condylar surface may provide the implant with a higher constraint during periods of low flexion and a lower constraint in periods of high flexion, providing more stability during standing and an increased range of motion during walking (i.e., during flexion and extension of the ankle).

Traditional ankle implants often use uniform condyle radii, which can restrict movement during high flexion and lead to discomfort or premature wear. The total ankle replacement implant described herein introduces a bicondylar talar component with distinct high constraint region for stability during low flexion (e.g., standing) and low constraint regions for increased flexibility during high flexion (e.g., walking), allowing for a range of motion that is similar to that of a natural ankle joint.

The total ankle replacement implant may include a tibial tray, stem, articulation component, and talar component. The talar component's bearing surface transitions smoothly between high and low constraint regions, defined by different radii, to replicate natural ankle kinematics. This approach aims to improve patient comfort, mobility, and implant longevity by balancing stability and motion across various positions of the ankle.

In certain embodiments, the total ankle replacement implant has a talar component and an articulation component configured to move relative to one another to facilitate movement of the patient's ankle after receiving a total ankle replacement implant. The talar component defines a convex bearing surface configured to be received within the concave articulating surface of the articulation component. In some embodiments, the talar component is bicondylar, having a lateral condyle defining a lateral bearing surface and a medial condyle defining a medial bearing surface. The articulation component may also have a lateral articulating surface and a medial articulating surface, configured to interface with the lateral and medial bearing surfaces of the talar component, respectively.

The bearing surface of the talar component may have a high constraint region and a low constraint region, where the high constraint and low constraint regions define the range of motion of the talar component relative to the articulation component. For example, the high constraint and low constraint regions may maximize contact between the bearing surface, and the articulation surface may be maximized when the ankle is in a neutral position (i.e., during standing), maximizing the stability of the total ankle replacement implant. When the ankle is in a flexed or extended position, contact between the bearing surface and the articulation surface may be minimized, providing the talar component with the ability to internally rotate or externally rotate relative to the articulation component.

Each condyle of the talar component, i.e., the lateral condyle and the medial condyle, may each have a high constraint region and a low constraint region. In this way, the talar component may be symmetrical in the lateral-medial direction. In some embodiments, each condyle may have a low constraint region at the anterior end and the posterior end of the talar component, such that the talar component is also symmetrical in the anterior-posterior direction.

In certain embodiments, the high constraint region(s) defines a first radius, or a distance between the bearing surface and the bottom side of the talar component, at a maximum height of the bearing surface relative to the bottom side of the talar component. The low constraint region(s) defines a second radius at a minimum height of the bearing surface relative to the bottom side of the talar component. The first radius of the high constraint region(s) may be greater than the second radius of the low constraint region(s). The difference between the first radius and the second radius creates the high constraint region(s) and low constraint region(s) that may increase the stability and the range of motion of the total ankle replacement implant, replicating the natural stability and range of motion of a biological ankle joint.

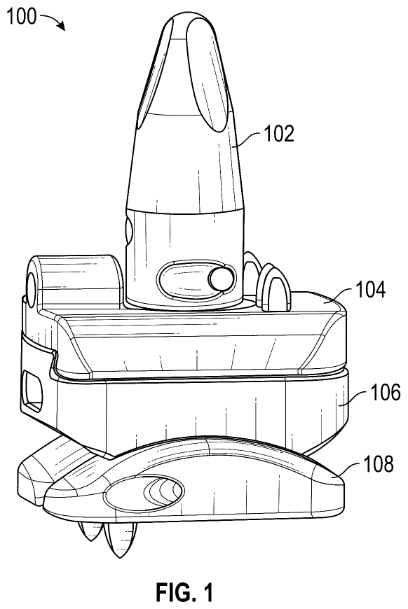

Referring now to FIG. 1, a total ankle replacement implant 100 may include the following components: a stem 102, a tibial tray 104, an articulation component 106, and a talar component 108. Each of the stem 102, tibial tray 104, and talar component 108 may be fabricated from a metallic material, such as titanium, a titanium alloy, stainless steel, cobalt chrome, a cobalt chrome alloy, or a combination thereof. In some embodiments, the stem 102, tibial tray 104, and talus component 108 are manufactured using 3D printing or additive manufacturing methods. In some embodiments, the articulating surface component 106 is formed of a biocompatible polymer, such as ultra-high-molecular-weight polyethylene (UHMWPE), a cross-linked polymer, a thermoplastic polymer such as polyether ether ketone (PEEK), or a combination thereof.

In embodiments, the stem 102, tibial tray 104, articulation component 106, and talar component 108 are assembled to form the total replacement implant 100 as shown in FIG. 1. When the total ankle replacement implant 100 is assembled within a patient's ankle joint, the stem 102 is placed within a canal resected within the patient's tibia, with the tibial tray 104 being attached to the stem 102 and the resected tibia at a distal end thereof. The articulation component 106 attaches to the distal end of the tibial tray 104, and is movably engaged with the talar component 108, which is fixed to the proximal end of the patient's resected talus bone.

The interface of the articulation component 106 and the talar component 108 may enable the articulation component 106 and the talar component 108 to move relative to one another, replicating the natural articulation or movement of a biological ankle joint. For example, in a biological ankle joint, the articulating surfaces of the tibia and the talus are responsible for the stability and the range of motion of the ankle joint. Similarly, in the total ankle replacement implant 100 described herein, the articulation component 106 and the talar component 108 interface in such a way, driven by the geometry of the talar component 108, that the implant 100 is stable in positions of low flexion and sufficiently flexible in positions of high flexion (i.e., when the foot and ankle are flexed or extended).

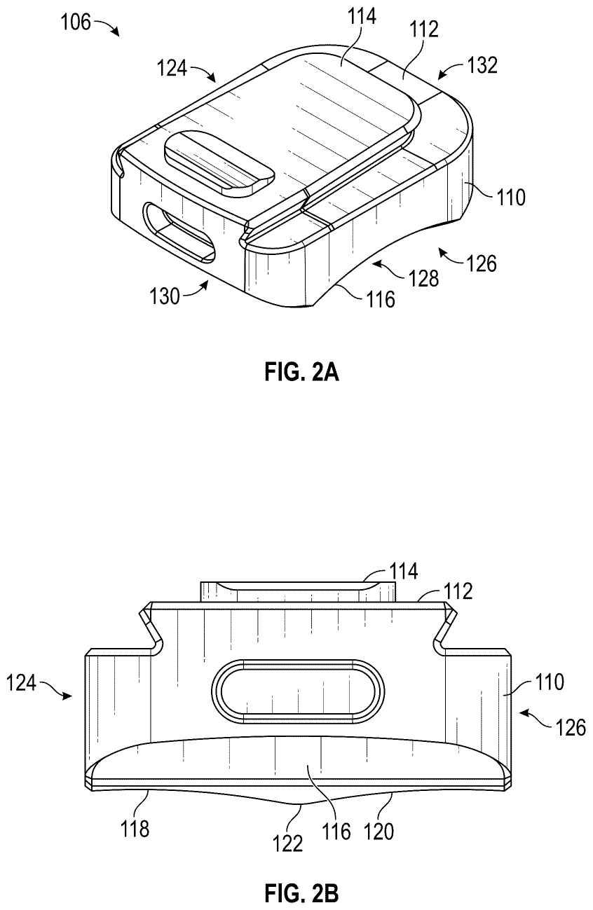

In embodiments, as shown in FIGS. 2A-2B, the articulation component 106 includes a substantially rectangular body 110 having an upper surface 112 configured to engage with the tibial tray 104. In some embodiments, the upper surface 112 has a projection 114 extending therefrom for securing the articulation component 106 to the tibial tray 104. For example, the projection 114 may be received by a reciprocal slot (not shown) within the tibial tray 104 for securing the tibial tray 104 and the articulation component 106 together in a fixed manner. The articulation component 106 may also have a lower articulation surface 116 opposite the body from the upper surface 112, where the lower articulation surface 116 is configured to interface with the talar component 108 to effectuate motion within the implant 100, mimicking the natural articulation of a biological ankle joint.

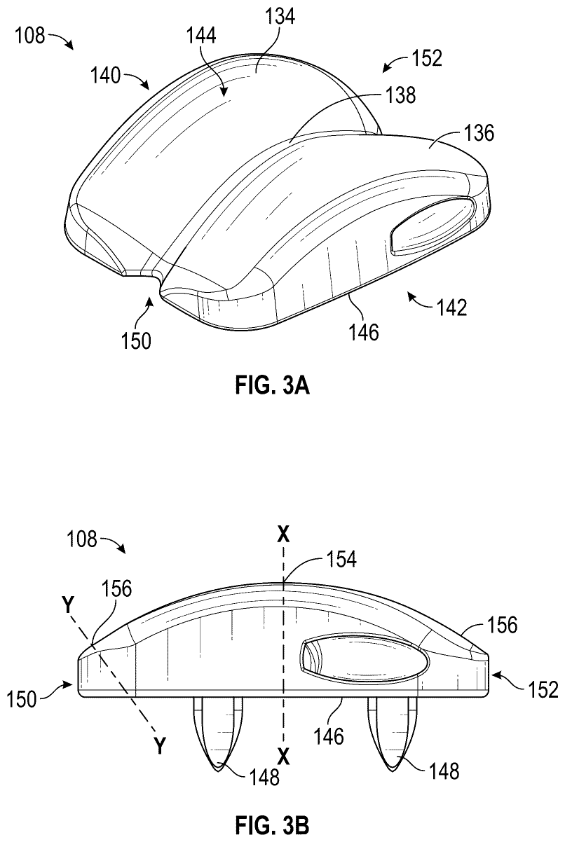

For example, the articulation surface 116 may include a lateral articulation surface 118 and a medial articulation surface 120, separated by a notch projection 122. The lateral articulation surface 118 is on a lateral side 124 of the articulation component 106, with the medial articulation surface 120 being on a medial side 126 of the implant, relative to the notch projection 122 which passes through the midline of the component body 110. The lateral articulation surface 118 and the medial articulation surface 120 may be configured to engage with the lateral and medial condyles, respectively, of the talar component 108, as shown and described in greater detail with respect to FIGS. 3A-3B. For example, both the lateral articulation surface 118 and the medial articulation surface 120 may define a radius of curvature 128 in the anterior-posterior direction of the body 110 for receiving the lateral and medial condyles of the talar component 108 to permit articulation of the articulation component 106 and the talar component 108 relative to each other. According to preferred embodiments, the radius of curvature 128 is a uniform radius of curvature, in which case the curvature of the talar component 108 defines the range of motion thereof when articulating relative to the anterior side 130 and posterior side 132 of the articulation component 106 during extension and flexion of the ankle. In some embodiments, the radius of curvature 128 is the same for both the lateral articulation surface 118 and the medial articulation surface 120, such that the articulation surface 116 is symmetrical on the lateral side 124 of the body 110 and the medial side 126 of the body 110 of the articulation component 106.

While the articulation component 106 has been shown and described as having a particular shape and with particular dimensions, one of ordinary skill in the art would recognize that the shape and/or size of the articulation component 106 may be changed without departing from the inventive concepts of the present disclosure. For example, the body 110 of the articulation component 106 may be any other suitable shape, other than substantially rectangular, for placement into the ankle joint. Similarly, the relative dimensions in the medial-lateral direction and the anterior-posterior direction may be modified or customized to accommodate the anatomy of a patient's ankle joint, so long as the radius of curvature 128 of the lateral articulation surface 118 and the medial articulation surface 120 remains unchanged.

Similarly, while certain features of the articulation component 106 have been described as being “lateral” or “medial” relative to the midline of the articulation component 106 or relative to the midline of a patient's body, in preferred embodiments, the articulation component 106 is symmetrical in the medial-lateral direction. As such, it would be understood that, while the articulation component 106 has been described relative to implantation in a patient's right ankle, the articulation component 106 as described herein could be used in a patient's right ankle or left ankle without any modifications thereto.

Referring now to FIGS. 3A-3B, the talar component 108 of the total ankle replacement implant 100 is shown and described in greater detail. In embodiments, the talar component 108 is bicondylar, having a lateral condyle 134 and a medial condyle 136, with a condylar notch 138 defined therebetween. The lateral condyle 134 may define the lateral articulation surfaced 118 on the lateral side 124 of the articulation component 106, and the medial condyle 136 may define the medial articulation surface 120 on the medial side 126 of the articulation component 106. The notch projection 122 may define the midline of the articulation component 106, relative to which the lateral side 124 and medial side 126 are defined. Together, the lateral condyle 134, medial condyle 136, and condylar notch 138 define an upper bearing surface 144 that engages with the lower articulation surface 116 of the articulation component 106 to effectuate flexion and extension of the ankle joint.

The talar component 108 may also include a bottom surface 146, opposite the upper bearing surface 144, that is substantially flat and configured for attachment to the patient's talus for securing the talar component 108 thereto. In some embodiments, as shown in FIG. 3B, the bottom surface 146 may include one or more anchors 148 extending in a downward direction to further secure the talar component 108 to the patient's talus.

In embodiments, the bearing surface 144 is a variable constraint articulating surface that provides a low constraint on articulation in the high flexion points and a high constraint on articulation in the low flexion points of the bearing surface 144. The variable constraint of the bearing surface 144 provides the total ankle replacement implant 100 with appropriate levels of constraint when needed due to the demands of the stress or flexion being placed on the bearing surface 144. For example, at low flexion angles (i.e., in a standing position) the surface contact between the bearing surface 144 and the articulating surface 116 of the articulation component 106 is maximized, necessitating high levels of constraint to maintain stability (i.e., to limit movement) of the articulation component 106 and the talar component 108. At high flexion angles (i.e., when maximally flexed or extended) the surface contact between the bearing surface 144 and the articulating surface 116 is minimized, and the low levels of constraint permit flexion and internal-external (IE) rotation of the articulation component 106 and the talar component 108 during any degree of flexion. The increased IE rotation may improve the kinematics of the total ankle replacement implant 100, providing the patient with a feeling of increased range of motion and mobility.

As shown in greater detail in FIG. 3B, the bearing surface 144 may include a high constraint region 154 at the upper most point of the bearing surface 144 and a low constraint region 156 at the lower most point of the bearing surface 144. For example, the high constraint region 154 may be between the anterior side 150 and posterior side 152 of the talar component 108, preferably at a center point 151 thereof. In the high constraint region 154, the radius (R1), or distance between the bottom surface 146 and the upper bearing surface 144 at the center point 151 of the talar component 108 in the anterior-posterior direction, is at a maximum. The low constraint region 156 may be at the anterior side 150 and/or posterior side 152 of the talar component 108, and in the low constraint region 156, the radius (R2), or distance between the bottom surface 146 and the upper bearing surface 144 at either the anterior end 150 or posterior end 152 of the talar component 108, is at a minimum.

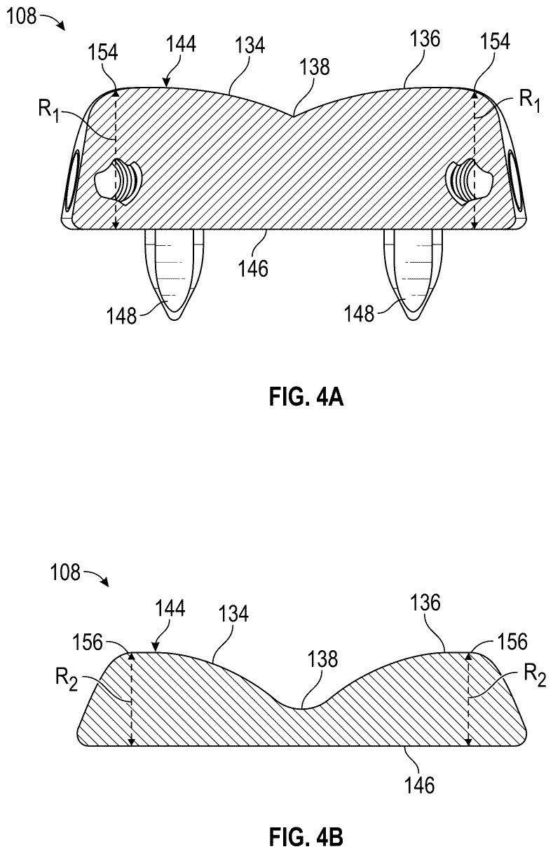

According to preferred embodiments, the talar component 108 is symmetrical in the anterior-posterior direction about the center point 151, such that each of the anterior end 150 and the posterior end 152 has a low constraint region 156, both having the same radius (R2). The radius, or distance between the bottom surface 146 and the upper bearing surface 144, may gradually and uniformly increase between the low constraint region 156 at the anterior end 150 of the talar component 108 and the high constraint region 154, and the radius may similarly decrease between the high constraint region 154 and the low constraint region 156 at the posterior end 152 of the talar component 108, forming a uniform and symmetrical arc across the upper bearing surface 144 of each of the lateral condyle 134 and the medial condyle 136. The talar component 108 may also be symmetrical in the lateral-medial direction, as shown in greater detail in FIGS. 4A-4B.

In embodiments, as shown in FIG. 4A, the lateral condyle 134 has a lateral high constraint region 154a and the medial condyle 136 has a medial high constraint region 154b. Each of the lateral high constraint region 154a and the medial high constraint region 154b have a radius (R1) that defines the distance between the bottom surface 146 and the upper bearing surface 144 at the high constraint regions 154a, 154b. In some embodiments, R1 is between about 0.5 inches to about 1 inch, such as about 0.75 inches.

In embodiments, as shown in FIG. 4B, the lateral condyle 134 has a lateral low constraint region 156a and the medial condyle 136 has a medial low constraint region 156b. Each of the lateral low constraint region 156a and the medial low constraint region 156b have a radius R2 that defines the distance between the bottom surface 146 and the upper bearing surface 144 at the low constraint regions 156a, 156b. In some embodiments, R2 is between about 0.25 inches and about 0.75 inches, such as about 0.5 inches or about 0.4 inches. In some embodiments, as shown and described with respect to FIG. 3B, the lateral condyle 134 may have two lateral low constraint regions 156a, with one on the anterior side 150 and one on the posterior side 152 of the talar component 108, and the medial condyle 134 may have two medial low constraint regions 156b, with one on the anterior side 150 and one on the posterior side 152 of the talar component 108.

While the talar component 108 has been shown and described as having a particular shape and with particular dimensions, one of ordinary skill in the art would recognize that the shape and/or size of the talar component 108 may be changed without departing from the inventive concepts of the present disclosure. For example, the relative dimensions in the medial-lateral direction and the anterior-posterior direction may be modified or customized to accommodate the anatomy of a patient's ankle joint, so long as R1 and R2 remain unchanged. Similarly, while certain features have been described as being “lateral” or “medial” relative to the midline of the talar component 108 or relative to the midline of a patient's body, in preferred embodiments, the talar component 108 is symmetrical in the medial-lateral direction. As such, it would be understood that, while the talar component 108 has been described relative to implantation in a patient's right ankle, the talar component 108 as described herein could be used in a patient's right ankle or left ankle without any modifications thereto.

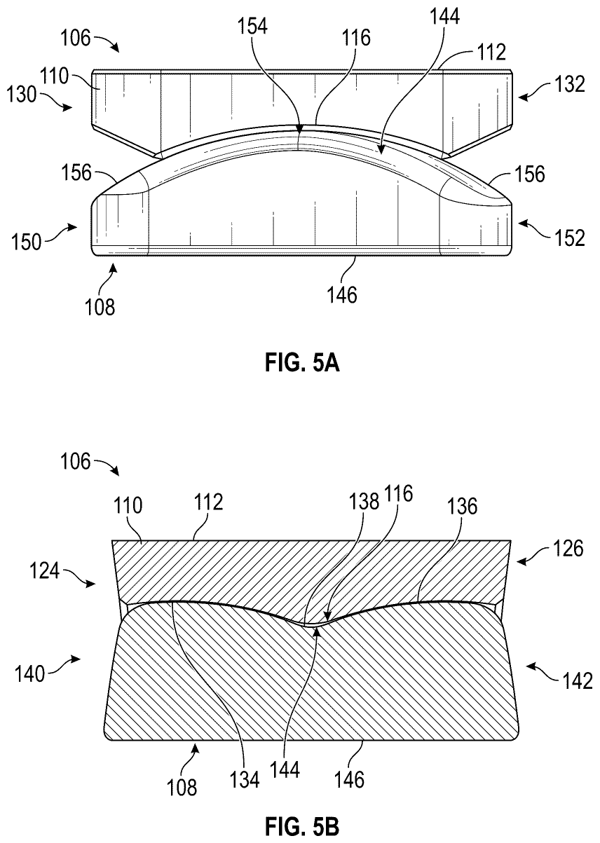

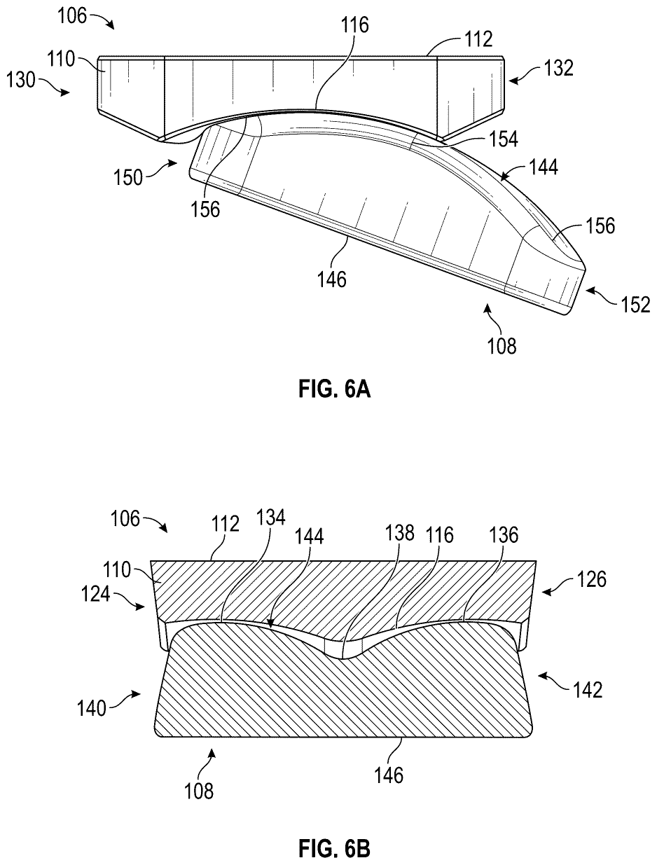

Referring now to FIGS. 5A-6B, the articulation component 106 and the talar component 108 are shown during various stages of articulation of the ankle. In embodiments, as shown in FIGS. 5A-5B, the articulation component 106 and the talar component 108 are in maximum contact with the highest constraint when the ankle is in a neutral or standing position. In embodiments, as shown in FIGS. 6A-6B, the articulation component 106 and the talar component 108 are in minimum contact with the lowest constraint when the ankle is in a maximally flexed position. The range of motion of the ankle, specifically, the ability of the articulation component 106 and the talar component 108 to internally and/or externally rotate relative to one another, may depend on the degree of contact between the articulation component 106 and the talar component 108.

In embodiments, as shown in FIGS. 5A-5B, when the articulation component 106 and the talar component 108 are in a neutral position, such as during standing, the contact between the articulation surface 116 of the articulation component 106 and the bearing surface 144 of the talar component 108 is maximized. With this contact between the articulation surface 116 and the bearing surface 144, the articulation component 106 and the talar component 108 are highly constrained, keeping the total ankle replacement implant 100 stable and limiting motion of the articulation component 106 and the talar component 108 relative to each other. For example, as shown in FIG. 5B, the contact between the articulation surface 116 and the bearing surface 144 may substantially or entirely prevent IE rotation between the articulation component 106 and the talar component 108.

In embodiments, as shown in FIGS. 6A-6B, when the ankle is completely flexed, the talar component 108 may shift posteriorly relative to the articulation component 106, as shown in FIG. 6A. When the ankle is flexed, the contact between the articulation surface 116 of the articulation component 106 and the bearing surface 144 of the talar component 108 is minimized and there is limited constraint on the articulation component 106 and the talar component 108, providing an increased range of motion, particularly internal and external rotational motion. For example, as shown in FIG. 6B, the articulating surface 116 and the bearing surface 144 are not in complete contact, which permits internal and/or external rotation between the articulation component 106 and the talar component 108.

While FIGS. 6A-6B depict the articulation component 106 and the talar component 108 during flexion of the ankle, it would be understood that the articulation component 106 and the talar component 108 behave similarly during extension of the ankle, with the talar component 108 shifted anteriorly relative to the articulation component 106 rather than posteriorly as shown in FIG. 6A.

While the disclosure has been described with reference to a number of exemplary embodiments, it would be understood by those skilled in the art that the disclosure is not limited to such embodiments. Rather, the disclosed embodiments can be modified to incorporate any number of variations, alterations, substitutions, or equivalent arrangements not described herein, but which are commensurate with the spirit and scope of the disclosure.

That which is claimed is:

1. A talar component for a total ankle replacement implant comprising:
a bearing surface comprising a high constraint region and a low constraint region,
wherein the high constraint region and the low constraint region of the bearing surface define a range of motion for the total ankle replacement implant.

2. The talar component of claim 1, wherein the high constraint region defines a first radius, the first radius being a distance between the bearing surface and an opposed bottom surface of the talar component at a maximum height of the bearing surface.

3. The talar component of claim 2, wherein the low constraint region defines a second radius, the second radius between the bearing surface and the opposed bottom surface of the talar component at a minimum height of the bearing surface.

4. The talar component of claim 3, wherein a ratio of the first radius to the second radius is between 2:1 to 1.5:1.

5. The talar component of claim 1, wherein the bearing surface is configured to maximize contact between the high constraint region of the bearing surface and an articulating surface configured to engage with and move relative to the bearing surface when the total ankle replacement is in a neutral position.

6. The talar component of claim 1, wherein the bearing surface is configured to minimize contact between the low constraint region of the bearing surface and an articulating surface configured to engage with and move relative to the bearing surface when the total ankle replacement is in a flexed position or an extended position.

7. The talar component of claim 6, wherein, in the flexed position or the extended position, the talar component is capable of internal rotation and external rotation relative to the articulation surface.

8. A total ankle replacement implant comprising:
an articulation component comprising an articulation surface; and
a talar component comprising a bearing surface;
wherein the bearing surface comprises a high constraint region and a low constraint region defining a range of motion of the talar component relative to the articulation component.

9. The total ankle replacement implant of claim 8, wherein the articulation surface is concave and defines a uniform radius of curvature.

10. The total ankle replacement implant of claim 9, wherein the bearing surface is convex and configured to be received within the articulation surface.

11. The total ankle replacement implant of claim 8, wherein the high constraint region defines a first radius, the first radius being a distance between the bearing surface and an opposed bottom surface of the talar component at a maximum height of the bearing surface, and wherein the low constraint region defines a second radius, the second radius being a distance between the bearing surface and the opposed bottom surface of the talar component at a minimum height of the bearing surface.

12. The total ankle replacement implant of claim 11, wherein the first radius is greater than the second radius.

13. The total ankle replacement implant of claim 8, wherein the bearing surface is configured to maximize contact between the high constraint region of the bearing surface and the articulating surface when the total ankle replacement is in a neutral position.

14. The total ankle replacement implant of claim 8, wherein the bearing surface is configured to minimize contact between the low constraint region of the bearing surface and the articulating surface when the total ankle replacement is in a flexed position or an extended position.

15. The total ankle replacement implant of claim 14, wherein, in the flexed position or the extended position, the talar component is capable of internal rotation and external rotation relative to the articulation surface.

16. The total ankle replacement implant of claim 8, wherein the talar component comprises a lateral condyle and a medial condyle, the lateral condyle defining a lateral bearing surface and the medial condyle defining a medial bearing surface.

17. The total ankle replacement implant of claim 16, wherein the articulation surface comprises a lateral articulation surface configured to receive the lateral bearing surface, and a medial articulation surface configured to receive the medial bearing surface.

18. A bearing surface of a talar component for a total ankle replacement implant, the bearing surface comprising a high constraint region defining a first radius and a low constraint region defining a second radius, wherein the first radius is a distance between the bearing surface and an opposed bottom surface of the talar component at a maximum height of the bearing surface, and wherein the second radius is a distance between the bearing surface and the opposed bottom surface of the talar component at a minimum height of the bearing surface.

19. The bearing surface of 18, wherein the first radius is greater than the second radius.

20. The bearing surface of claim 18, wherein the high constraint region and the low constraint region define a range of motion of the total ankle replacement implant.
